(19) United States
(12) Patent Application Publication
(10) Pub. No.: US 2009/0298135 A1
(43) Pub. Date: Dec. 3, 2009

(54) Method for fermentative production of L-methionine

(75) Inventors: Thomas Maier, Dachau (DE); Christoph Winterhalter, Pocking (DE); Kerstin Pfeiffer, Munchen (DE)

Correspondence Address: COLLARD & ROE, P.C., 1077 NORTHERN BOULEVARD, ROSLYN, NY, 11576, US

(73) Assignee: Consortium fur Elektrochemische Industrie GmbH

(21) Appl. No.: 10/768,528

(22) Filed: Jan. 30, 2004

(30) Foreign Application Priority Data
Feb. 6, 2003 (DE) 103 05 774.9

(51) Int. Cl. C12P 13/12; C12N 1/21; C12N 1/15; C12N 1/19; C12N 15/63

(52) U.S. Cl. 435/113; 435/252.3; 435/252.33; 435/254.1; 435/255.1; 435/320.1

(57) ABSTRACT

A microorganism strain suitable for fermentative production of L-methionine and preparable from a starting strain, which comprises increased activity of a yjeH gene product or of a gene product of a yjeH homolog, compared to the starting strain.

### BACKGROUND OF THE INVENTION

1. Field of the Invention

The invention relates to a method for producing L-methionine by means of fermentation.

2. The Prior Art

The amino acid methionine plays an outstanding part in animal feeding. Methionine is one of the essential amino acids that cannot be biosynthetically produced in the metabolism of vertebrates. Consequently, in animal breeding, intake of sufficient quantities of methionine with the feed is essential. However, since the amounts of methionine present in traditional feed plants (such as soya or cereals) are often too low for ensuring optimal animal feeding (particularly for pigs and poultry), it is advantageous to admix methionine as an additive to the animal feed. The great importance of methionine for animal feeding can also be attributed to the fact that, apart from L-cysteine (or L-cystine), methionine is the crucial sulfur source in the metabolism. Although the animal metabolism can convert methionine to cysteine, it cannot do so vice versa.

In the prior art, methionine is produced by chemical synthesis on the scale of >100,000 metric tons per year. In this process, first acrolein and methyl mercaptan are reacted to give 3-methylthiopropionaldehyde which in turn, together with cyanide, ammonia and carbon monoxide, gives hydantoin which can ultimately be hydrolyzed to give a racemate, an equimolar mixture of the two stereoisomers D- and L-methionine. Since the L-form is the only biologically active form of the molecule, the D-form present in the feed must first be converted to the active L-form by metabolic Des- and transamination.

Although methods are known which allow production of enantiomerically pure L-methionine by resolution of the racemate or by means of hydantoinases, these methods have so far not been introduced to the animal feed industry, due to high costs.

In a clear contrast to methionine, most of the other natural, proteinogenic amino acids are produced primarily by fermentation of microorganisms. Here the availability of appropriate biosynthetic pathways or synthesizing these natural amino acids in microorganisms is utilized. Moreover, many fermentation methods achieve very low production costs by using inexpensive reactants such as glucose and mineral salts and moreover provide the biologically active L-form of the amino acid in question.

However, biosynthetic pathways of amino acids in wildtype strains are subject to a tight metabolic control which ensures that the amino acids are produced only for the cell's own use. An important requirement for efficient production processes is therefore the availability of suitable microorganisms which, in contrast to wildtype organisms, have a drastically increased production of the desired amino acid.

Amino acid-overproducing microorganisms of this kind may be generated by traditional mutation/selection methods and/or by modern, specific, recombinant techniques (metabolic engineering). In the latter, firstly genes or alleles are identified which cause amino acid overproduction, due to their modification, activation or inactivation. These genes/alleles are then introduced into a microorganism strain or are inactivated, using molecular-biological techniques, so that optimal overproduction is achieved. Frequently, however, only the combination of several, different measures results in a truly efficient production.

The biosynthesis of L-methionine in microorganisms is very complex. The amino acid body of the molecule is derived from L-aspartate which is converted to L-homoserine via aspartylsemialdehyde/aspartyl phosphate. This is followed by three enzymic steps which involve replacing (via O-succinyl homoserine and cystathionine) the hydroxyl group on the molecule with a thiol group, the latter being mobilized from a cysteine molecule, resulting in homocysteine. In the final step of the biosynthesis, L-methionine is finally produced by methylation of the thiol group. The methyl group derives from the serine metabolism.

Formally, methionine is thus synthesized for its part in the microbial metabolism from the amino acids aspartate, serine and cysteine and therefore requires a highly complex biosynthesis, compared to other amino acids. In addition to the main synthetic pathway (aspartate-homoserine-homocysteine), cysteine biosynthesis and thus the complex fixation of inorganic sulfur and also the C1 metabolism must also be optimally coordinated.

For these reasons, the fermentative production of L-methionine has not been worked on very intensively in the past. In recent years, however, decisive progress has been made in the optimization of the serine and cysteine metabolisms so that fermentative production of L-methionine now appears realistic. Consequently, first studies in this direction have recently been described in the prior art.

For fermentative production of L-methionine, the following genes/alleles whose use can result in L-methionine overproduction are known in the prior art:

metA alleles as described in an application by the same applicant from Nov. 10, 2002 or in Japanese Patent No. JP2000139471A. These metA alleles code for O-homoserine transsuccinylases which are subject to a reduced feedback inhibition by L methionine. This leads to extensive decoupling of the formation of O-succinylhomoserine from the cellular methionine level.

metJ deletion as described in Japanese Patent No. JP2000139471A. The metJ gene codes for a central gene regulator of methionine metabolism and thus plays a crucial role in the control of methionine biosynthesis gene expression.

The prior art likewise suggests that known measures ensuring an improved synthesis of L-serine and L-cysteine have a positive influence on L-methionine production.

### SUMMARY OF THE INVENTION

It is the object of the present invention to provide a microorganism strain which makes L-methionine overproduction possible. Another object is to provide a method for producing L-methionine by means of the microorganism strain of the invention.

The first object is achieved by a microorganism strain preparable from a starting strain, which has an increased activity of the yjeH gene product or of a gene product of a yjeH homolog, compared to the starting strain.

In accordance with the present invention, the activity of the yjeH gene product is also increased when the total activity in the cell is increased due to an increase in the amount of gene product in the cell, and the activity of the yjeH gene product per cell is increased, although the specific activity of the gene product remains unchanged.

The Escherichia coli yjeH gene was identified as open reading frame in the course of sequencing of the genome (Blattner et al. 1997, Science 277:1453-1462) and codes for a protein of 418 amino acids. Up until now, it has not been possible to assign any physiological function to the yjeH gene. A database search for proteins with sequence homology (FASTA algorithm of GCG Wisconsin Package, Genetics Computer Group (GCG) Madison, Wis.) also provides few clues, since significant similarities are indicated only to proteins whose function is likewise unknown.

The yjeH gene and the yjeH gene product (YjeH protein) are characterized by the sequences SEQ ID No. 1 and SEQ ID No. 2, respectively. yjeH homologs are to be understood as meaning, within the scope of the present invention, those genes whose sequences are more than 30%, preferably more than 53%, identical in an analysis using the BESTFIT algorithm (GCG Wisconsin Package, Genetics Computer Group (GCG) Madison, Wisconsin). Particular preference is given to sequences which are more than 70% identical.

Likewise, YjeH-homologous proteins are to be understood as meaning proteins whose sequences are more than 30% (BESTFIT algorithm (GCG Wisconsin Package, Genetics Computer Group (GCG) Madison, Wis.)), and preferably more than 53%, identical. Particular preference is given to sequences which are more than 70% identical.

Thus, yjeH homologs also mean allele variants of the yjeH gene, in particular functional variants, which are derived from the sequence depicted in SEQ ID No. 1 by deletion, insertion or substitution of nucleotides, but with the enzymic activity of the particular gene product being retained.

Microorganisms of the invention which have increased activity of the yjeH gene product, compared to the starting strain, may be generated using standard molecular-biological techniques.

Suitable starting strains are in principle any organisms which have the biosynthetic pathway for L-methionine, are accessible to recombinant methods and can be cultured by fermentation. Microorganisms of this kind may be fungi, yeasts or bacteria. Preferred bacteria are those of the phylogenetic group of eubacteria. Particular preference is given to microorganisms of the family Enterobacteriaceae and in particular of the species Escherichia coli.

The increase in activity of the yjeH gene product in the microorganism of the invention is achieved, for example, by enhanced expression of the yjeH gene. This may involve an increased copy number of the yjeH gene in a microorganism and/or increased expression of the yjeH gene, due to suitable promoters. Increased expression preferably means that the yjeH gene is expressed at least twice as strong as in the starting strain.

The copy number of the yjeH gene in a microorganism may be increased using methods known to someone skilled in the art. Thus, for example, the yjeH gene may be cloned into plasmid vectors having multiple copies per cell (e.g. pUC19, pBR322, pACYC184 for Escherichia coli) and introduced into the microorganism. Alternatively, multiple copies of the yjeH gene may be integrated into the chromosome of a microorganism. Integration methods which may be used are the known systems with temperate bacteriophages, integrative plasmids or integration via homologous recombination (e.g. Hamilton et al., 1989, J. Bacteriol. 171: 4617-4622).

Preference is given to increasing the copy number by cloning a yjeH gene into plasmid vectors under the control of a promoter. Particular preference is given to increasing the copy number in Escherichia coli by cloning a yjeH gene in a pACYC derivative such as, for example, pACYC184-LH (deposited according to the Budapest Treaty with the Deutsche Sammlung fur Mikroorganismen und Zellkulturen, Brunswick, Germany on 8.18.95 under the number DSM 10172).

A control region for expressing a plasmid-encoded yjeH gene, which may be used, is the natural promoter and operator region.

Enhanced expression of a yjeH gene, however, may also be carried out by means of other promoters. Appropriate promoter systems such as, for example, the constitutive GAPDH promoter of the gapA gene or the inducible lac, tac, trc, lambda, ara or tet promoters in Escherichia coli are known to the skilled worker (Makrides S. C., 1996, Microbiol. Rev. 60: 512-538). Such constructs may be used in a manner known per se on plasmids or chromosomally.

Furthermore, enhanced expression may be achieved by translation start signals such as, for example, the ribosomal binding site or start codon of the gene being present in an optimized sequence on the particular construct or by replacing codons which are rare according to “codon usage” with more frequently occurring codons.

Microorganism strains having the modifications mentioned are preferred embodiments of the invention.

A yjeH gene is cloned into plasmid vectors, for example, by specific amplification via the polymerase chain reaction using specific primers which cover the complete yjeH gene and subsequent ligation with vector DNA fragments.

Preferred vectors used for cloning a yjeH gene are plasmids which already contain promoters for enhanced expression, for example the constitutive GAPDH promoter of the Escherichia coli gapA gene.

The invention thus also relates to a plasmid which comprises a yjeH gene with a promoter.

Furthermore, particular preference is given to vectors which already contain a gene/allele whose use results in a reduced feedback inhibition of the L-methionine metabolism, such as a mutated metA allele, for example (described in application DE A-10247437). Such vectors enable inventive microorganism strains with high amino acid overproduction to be directly prepared from any microorganism strain, since such a plasmid also reduces feedback inhibition of the methionine metabolism in a microorganism.

The invention thus also relates to a plasmid which comprises a genetic element for deregulating the methionine metabolism and a yjeH gene with a promoter.

Using a common transformation method (e.g. electroporation), the yjeH-containing plasmids are introduced into microorganisms and selected, for example, by means of antibiotic resistance to plasmid-carrying clones.

The invention thus also relates to methods for preparing a microorganism strain of the invention, which comprise introducing a plasmid of the invention into a starting strain.

Particularly preferred strains for the transformation with plasmids of the invention are those whose chromosomes already have alleles which may likewise favor L-methionine production, such as, for example,

a metJ deletion (as described in JP2000139471A) or

alleles effecting improved serine provision, such as feedback-resistant serA variants (as described, for example, in EP0620853B1 or EP0931833A2)

or genes effecting improved cysteine provision, such as feedback-resistant cysE variants (as described, for example, in WO 97/15673).

Production of L-methionine is carried out with the aid of a microorganism strain of the invention in a fermenter according to known methods.

The invention thus also relates to a method for producing L methionine, which comprises using a microorganism strain of the invention in a fermentation and removing the L-methionine produced from the fermentation mixture.

The microorganism strain is grown in the fermenter in continuous culture, in batch culture or, preferably, in fed-batch culture. Particular preference is given to continuously metering in a carbon source during fermentation.

Preferred carbon sources used are sugars, sugar alcohols or organic acids. Particular preference is given to using glucose, lactose or glycerol as carbon sources in the method according to the invention.

Preferably, the carbon source is metered in so as to ensure that the carbon source content in the fermenter is maintained in a range from 0.1-50 g/l during fermentation, particular preference being given to a range from 0.5-10 g/l.

Preferred nitrogen sources used in the method of the invention are ammonia, ammonium salts and protein hydrolysates. When using ammonia for correcting the pH stat, this nitrogen source is metered in in regular intervals during fermentation.

Further media additives which may be added are salts of the elements phosphorus, chlorine, sodium, magnesium, nitrogen, potassium, calcium, iron and, in traces (i.e. in μM concentrations), salts of the elements molybdenum, boron, cobalt, manganese, zinc and nickel.

It is also possible to add organic acids (e.g. acetate, citrate), amino acids (e.g. leucine) and vitamins (e.g. B₁, B₁₂) to the medium.

Complex nutrient sources which may be used are, for example, yeast extract, corn steep liquor, soybean meal or malt extract.

The incubation temperature for mesophilic microorganisms is preferably 15-45° C., particular preferably 30-37° C.

The fermentation is preferably carried out under aerobic growth conditions. Oxygen is introduced into the fermenter by means of compressed air or by means of pure oxygen.

During fermentation, the pH of the fermentation medium is preferably in the range from pH 5.0 to 8.5, particular preference being given to pH 7.0.

A sulfur source may be fed in during fermentation for production of L-methionine. Preference is given here to using sulfates or thiosulfates.

Microorganisms fermented according to the method described secrete in a batch or fed-batch process, after a growing phase, L-methionine into the culture medium over a period of time from 10 to 150 hours.

The L-methionine produced may be obtained from fermenter broths via suitable measures for amino acid isolation (e.g. ion exchange methods, crystallization, etc.).

### DETAILED DESCRIPTION OF THE PREFERRED EMBODIMENT

The following examples serve to further illustrate the invention. The strain W3110ΔJ/pKP450 was deposited as a bacterial strain having an inventive plasmid with yjeH gene and suitable for L-methionine production according to the invention with the DSMZ (Deutsche Sammlung fur Mikroorganismen und Zellkulturen GmbH, D-38142 Brunswick, Germany) under the number DSM 15421 according to the Budapest Treaty.

### EXAMPLE 1

### Cloning of the Basic Vector pKP228

In order to place the yjeH gene under the control of a constitutive promoter, first a basic vector containing the constitutive GAPDH promoter of the gapA gene for Escherichia coli glyceraldehyde 3-dehydrogenase was constructed. To this end, a polymerase chain reaction using the primers

GAPDHfw:

(SEQ. ID. NO: 3)

5′ GTC GAC GCG TGA GGC GAG TCA GTC GCG TAA TGC 3′
Mlu I
GAPDHrev1:

(SEQ. ID. NO: 4)

5′ GAC CTT AAT TAA GAT CTC ATA TAT TCC ACC AGC TAT
TTG TTA G 3′
Pac I Bgl II

and chromosomal DNA of E. coli strain W3110 (ATCC27325) was carried out. The resulting DNA fragment was purified with the aid of an agarose gel electrophoresis and subsequently isolated (Qiaquick Gel Extraction Kit, Qiagen, Hilden, D). Thereafter, the fragment was treated with the restriction enzymes PacI and MluI and cloned into the vector pACYC184-LH, likewise cleaved with PacI/MluI (deposited according to the Budapest Treaty with the Deutsche Sammlung fur Mikroorganismen und Zellkulturen, Brunswick on 8.18.95 under the number DSM 10172). The new construct was referred to as pKP228.

### EXAMPLE 2

### Cloning of the yjeH Gene

The yjeH gene from Escherichia coli W3110 strain was amplified with the aid of the polymerase chain reaction. The

oligonucleotides

(SEQ. ID. NO: 5)

| | |
|---|---|
| yjeH-fw: | 5′-ATT GCT GGT TTG CTG CTT-3′ |
| and | |

(SEQ. ID. NO: 6)

| | |
|---|---|
| yjeH-rev: | 5′-AGC ACA AAA TCG GGT GAA-3′ |

were used as specific primers and chromosomal DNA of the E. coli strain W3110 (ATCC27325) was used as template. The resulting DNA fragment was purified and isolated by agarose gel electrophoresis (Qiaquick Gel Extraction Kit, Qiagen, Hilden, Germany). Cloning was carried out by way of blunt end ligation with a BglII-cleaved pKP228 vector whose 5′-protruding ends were filled in using Klenow enzyme. The procedure stated places the yjeH gene downstream of the GAPDH promoter in such a way that transcription can be initiated therefrom. The resulting vector is referred to as pKP450.

### EXAMPLE 3

### Combination of the yjeH Gene with a Feedback-Resistant metA Allele

A metA allele which is described in the patent application DE A-10247437 of Nov. 10, 2002 and which codes for a feedback-resistant O-homoserine transsuccinylase was amplified by polymerase chain reaction using the template pKP446 (likewise described in the patent application DE A-10247437) and the primers

(SEQ. ID. NO: 7)

| | |
|---|---|
| metA-fw | 5′-CGC CCA TGG CTC CTT TTA GTC ATT CTT-3′ |
| | NcoI |

(SEQ. ID. NO: 8)

| | |
|---|---|
| metA-rev | 5′-CGC GAG CTC AGT ACT ATT AAT CCA GCG-3′ |
| | SacI. |

In the process, terminal cleavage sites for restriction endonucleases NcoI and SacI were generated. The DNA fragment obtained was digested with the same endonucleases, purified and cloned into the NcoI/SacI-cleaved pKPA50 vector. The resulting plasmid was referred to as pKP451.

In order to prepare a control plasmid containing the metA allele but not the yjeH gene, the yjeH gene was deleted from pKP451. For this purpose, pKP451 was cleaved with Ec1136II and PacI, the protruding ends were digested off with Klenow enzyme and the vector was religated. The plasmid obtained in this way is referred to as pKP446AC.

### EXAMPLE 4

### Generation of a Chromosomal metJ Mutation

The genes metJ/B were amplified by polymerase chain reaction using the primers

metJ-fw:

(SEQ. ID. NO: 9)

5′-GAT CGC GGC CGC TGC AAC GCG GCA TCA TTA AAT TCG
A-3′
and
metJ-rev:

(SEQ. ID. NO: 10)

5′-GAT CGC GGC CGC AGT TTC AAC CAG TTA ATC AAC
TGG-3′

and chromosomal DNA from Escherichia coli W3110 (ATCC27325).

The fragment comprising 3.73 kilobases was purified, digested with the restriction endonuclease NotI and cloned into the NotI-cleaved pACYC184-LH vector (see example 1). This was followed by inserting a kanamycin resistance cassette into the metJ gene at the internal AflIII-cleavage site. To this end, a digestion with AflIII was followed by generating blunt ends using Klenow enzyme. The kanamycin cassette in turn was obtained from the vector pUK4K (Amersham Pharmacia Biotech, Freiburg, Germany) by PvuII restriction and inserted into the metJ gene via ligation. The metj::kan cassette was then obtained as linear fragment from the thus prepared pKP440 vector by NotI restriction and chromosomally integrated into the recBC/sbcB strain JC7623 (E.coli Genetic Stock Center CGSC5188) according to the method of Winans et al. (J. Bacteriol. 1985, 161:1219-1221). In a final step, the metj::kan mutation was finally transduced by P1 transduction (Miller, 1972, Cold Spring Harbour Laboratory, New York, pp. 201-205) into the W3110 (ATCC27325) wildtype strain, thus generating the strain W3110ΔJ.

After verifying the metj::kan insertion, the W3110ΔJ strain was transformed in each case either with the yjeH-carrying plasmids or the control plasmids, followed by selecting corresponding transformants with tetracycline.

### EXAMPLE 5

### Producer Strain Precultures for Fermentation

A preculture for the fermentation was prepared by inoculating 20 ml of LB medium (10 g/l tryptone, 5 g/l yeast extract, 10 g/l NaCl), which additionally contained 15 mg/l tetracycline, with the producer strains and incubation in a shaker at 150 rpm and 30° C. After seven hours, the entire mixture was transferred into 100 ml of SM1 medium (12 g/l K₂HPO₄; 3 g/l KH₂PO₄; 5 g/l (NH₄)₂SO₄; 0.3 g/l MgSO₄×7 H₂O; 0.015 g/l CaCl₂×2 H₂O; 0.002 g/l FeSO₄×7 H₂O; 1 g/l Na₃citrate×2 H₂O; 0.1 g/l NaCl; 1 ml/l trace element solution comprising 0.15 g/l Na₂MoO₄×2 H₂O; 2.5 g/l Na₃BO₃; 0.7 g/l CoCl₂×6 H₂O; 0.25 g/l CuSO₄×5 H₂O; 1.6 g/l MnCl₂×4 H₂O; 0.3 g/l ZnSO₄×7 H₂O), supplemented with 5 g/l glucose; 0.5 mg/l vitamin B₁ and 15 mg/l tetracycline. Further incubation was carried out at 30° C. and 150 rpm for 17 hours.

### Example 6

### Fermentative Production of L-Methionine

The fermenter used was a Biostat B instrument from Braun Biotech (Melsungen, Germany), which has a maximum culture volume of 2 l. The fermenter containing 900 ml of SM1 medium supplemented with 15 g/l glucose, 10 g/l tryptone, 5 g/l yeast extract, 3 g/l Na₂S₂O₃×5H₂O, 0.5 mg/l vitamin B₁, 30 mg/l vitamin B₁₂ and 15 mg/l tetracycline was inoculated with the preculture described in example 5 (optical density at 600 nm: approx. 3). During fermentation, the temperature was adjusted to 32° C. and the pH was kept constant at pH 7.0 by metering in 25% ammonia. The culture was gassed with sterilized compressed air at 5 vol/vol/min and stirred at a rotational speed of 400 rpm. After oxygen saturation had decreased to a value of 50%, the rotational speed was increased to up to 1 500 rpm via a control device in order to maintain 50% oxygen saturation (determined by a pO₂ probe calibrated to 100% saturation at 900 rpm). As soon as the glucose content in the fermenter had decreased from initially 15 g/l to approx. 5-10 g/l, a 56% glucose solution was metered in. The feeding took place at a flow rate of 6-12 ml/h and the glucose concentration in the fermenter was kept constant between 0.5-10 g/l. Glucose was determined using the glucose analyzer from YSI (Yellow Springs, Ohio, USA). The fermentation time was 48 hours, after which samples were taken and the cells were removed from the culture medium by centrifugation. The resulting culture supernatants were analyzed by reversed phase HPLC on a LUNA 5 μ C18(2) column (Phenomenex, Aschaffenburg, Germany) at a flow rate of 0.5 ml/min. The eluent used was diluted phosphoric acid (0.1 ml of conc. phosphoric acid/l). Table 1 shows the L-methionine contents obtained in the culture supernatant.

TABLE 1

| Strain | Genotype (plasmid) | L-Methionine [g/l] |
|---|---|---|
| W3110ΔJ/pKP228 | — | <0.1 g/l |
| W3110ΔJ/pKP450 | yjeH | 0.8 g/l |
| W3110ΔJ/pKP451 | metAfbr yjeH | 4.8 g/l |
| W3110ΔJ/pKP446AC | metAfbr | 0.9 g/l |

fbr: feedback-resistant

Accordingly, while only a few embodiments of the present invention have been shown and described, it is obvious that many changes and modifications may be made thereunto without departing from the spirit and scope of the invention.

What is claimed is:

1. A microorganism strain suitable for fermentative production of L-methionine and prepared from a starting strain, said microorganism strain comprising increased activity of a yjeH gene product or of a gene product of a yjeH homolog, compared to said starting strain.

2. The microorganism strain as claimed in claim 1, wherein said microorganism strain is a fungus, a yeast or a bacterium.

3. The microorganism strain as claimed in claim 2, wherein the microorganism strain is a bacterium of the family Enterobacteriaceae.

4. The microorganism strain as claimed in claim 3, wherein the microorganism strain is a bacterium of the species Escherichia coli.

5. The microorganism strain as claimed in claim 1, wherein a copy number of the yjeH gene in the microorganism is increased or expression of said yjeH gene has been increased by using suitable promoters or translation signals.

6. The microorganism strain as claimed in claim 5, wherein the promoter is selected from the group consisting of constitutive GAPDH promoter of the gapA gene, inducible lac, tac, trc, lambda, ara and tet-promoters.

7. The microorganism strain as claimed in claim 1, wherein said microorganism strain is an Escherichia coli strain in which the increased activity of a yjeH gene product is based on increasing a copy number of the yjeH gene in a pACYC derivative.

8. A plasmid comprising a yjeH gene with a promoter.

9. The plasmid as claimed in claim 8, said plasmid additionally recruiting a genetic element for deregulating methionine metabolism.

10. A method for preparing from a starting strain a microorganism strain suitable for fermentative production of L-methionine, said microorganism strain comprising increased activity of a yjeH gene product or of a gene product of a yjeH homolog, compared to said starting strain, said method comprising introducing a plasmid into said starting strain, the plasmid comprising a yjeH gene with a promoter.

11. A method for preparing L-methionine comprising using a microorganism strain in a fermentation and removing L-methionine from the fermentation mixture, wherein said microorganism strain is suitable for fermentative production of L-methionine and preparable from a starting strain, said microorganism strain comprising increased activity of a yjeH gene product or of a gene product of a yjeH homolog, compared to said starting strain.

12. The method as claimed in claim 11, wherein the microorganism strain is grown as continuous culture, as batch culture or as fed-batch culture in a fermenter.

13. The method as claimed in claim 11, wherein a carbon source is continuously metered in during fermentation.

14. The method as claimed in claim 13, wherein the carbon source is sugar, sugar alcohols or organic acids.

15. The method as claimed in claim 13, wherein the carbon source is metered in so as to ensure that the carbon source content in the fermenter is maintained within a range from 0.1-50 g/l during fermentation.

16. The method as claimed in claim 11, wherein ammonia, ammonium salts or protein hydrolyzates are used as a nitrogen source during fermentation.

17. The method as claimed in claim 11, wherein the fermentation is carried out under aerobic growth conditions.


SEQUENCE LISTING (10 sequences)

SEQ ID NO: 1 - DNA, 1652 bp, Escherichia Coli
  CDS (141)..(1394)
agcacaaaatcgggtgaaaaccctgattcacctcacatttcatcgcaatttcttcatcgccgtcataagcgaatctgattgtgctaccatcgaaaatctacgcagttgcccaaaatttgggcgcaatcggacatcaacccatgagtggactcaaacaagaactggggctggcccagggcattggcctgctatcgacgtcattattaggcactggcgtgtttgccgttcctgcgttagctgcgctggtagcgggcaataacagcctgtgggcgtggcccgttttgattatcttagtgttcccgattgcgattgtgtttgcgattctgggtcgccactatcccagcgcaggcggcgtcgcgcacttcgtcggtatggcgtttggttcgcggcttgagcgagtcaccggctggctgtttttatcggtcattcccgtgggtttgcctgccgcactacaaattgccgccgggttcggccaggcgatgtttggctggcatagctggcaactgttgttggcagaactcggtacgctggcgctggtgtggtatatcggtactcgcggtgccagttccagtgctaatctacaaaccgttattgccggacttatcgtcgcgctgattgtcgctatctggtgggcgggcgatatcaaacctgcgaatatcccctttccggcacctggtaatatcgaacttaccgggttatttgctgcgttatcagtgatgttctggtgttttgtcggtctggaggcatttgcccatctcgcctcggaatttaaaaatccagagcgtgattttcctcgtgctttgatgattggtctgctgctggcaggattagtctactggggctgtacggtagtcgtcttacacttcgacgcctatggtgaaaaaatggcggcggcagcatcgcttccaaaaattgtagtgcagttgttcggtgtaggagcgttatggattgcctgcgtgattggctatctggcctgctttgccagtctcaacatttatatacagagcttcgcccgcctggtctggtcgcaggcgcaacataatcctgaccactacctggcacgcctctcttctcgccatatcccgaataatgccctcaatgcggtgctcggctgctgtgtggtgagcactttggtgattcatgctttagagatcaatctggacgctcttattatttatgccaatggcatctttattatgatttatctgttatgcatgctggcaggctgtaaattattgcaaggacgttatcgactactggcggtggttggcgggctgttatgcgttctgttactggcaatggtcggctggaaaagtctctatgcgctgatcatgctggcggggttatggctgttgctgccaaaacgaaaaacgccggaaaatggcataaccacataatccggcgtttcgacattaatcctggcgatcgtctttatgatcaaggcggtcgcgctcatcatcctttcgctggtactcaccatcaaaagtattaccgccaccggtcccggcgctaaaaccgccgccaggcatgcgagaaaagcgcaaatgcggcatcaacttcactgtcagatgcttttgcaccggcggcaataaaagtagaagaccgaggaagtcggtaaaaaagcccggtaataaaagcagcaaaccagcaat

SEQ ID NO: 2 - protein, 418 aa, Escherichia Coli
MSGLKQELGLAQGIGLLSTSLLGTGVFAVPALAALVAGNNSLWAWPVLIILVFPIAIVFAILGRHYPSAGGVAHFVGMAFGSRLERVTGWLFLSVIPVGLPAALQIAAGFGQAMFGWHSWQLLLAELGTLALVWYIGTRGASSSANLQTVIAGLIVALIVAIWWAGDIKPANIPFPAPGNIELTGLFAALSVMFWCFVGLEAFAHLASEFKNPERDFPRALMIGLLLAGLVYWGCTVVVLHFDAYGEKMAAAASLPKIVVQLFGVGALWIACVIGYLACFASLNIYIQSFARLVWSQAQHNPDHYLARLSSRHIPNNALNAVLGCCVVSTLVIHALEINLDALIIYANGIFIMIYLLCMLAGCKLLQGRYRLLAVVGGLLCVLLLAMVGWKSLYALIMLAGLWLLLPKRKTPENGITT

SEQ ID NO: 3 - DNA, 33 bp, Artificial Sequence
  Description of Artificial Sequence GAPDHfw
gtcgacgcgtgaggcgagtcagtcgcgtaatgc

SEQ ID NO: 4 - DNA, 43 bp, Artificial Sequence
  Description of Artificial Sequence GAPDHrev1
gaccttaattaagatctcatatattccaccagctatttgttag

SEQ ID NO: 5 - DNA, 18 bp, Artificial Sequence
  Description of Artificial Sequence yjeH-fw
attgctggtttgctgctt

SEQ ID NO: 6 - DNA, 18 bp, Artificial Sequence
  Description of Artificial Sequence yjeH-rev
agcacaaaatcgggtgaa

SEQ ID NO: 7 - DNA, 27 bp, Artificial Sequence
  Description of Artificial Sequence metA-fw
cgcccatggctccttttagtcattctt

SEQ ID NO: 8 - DNA, 27 bp, Artificial Sequence
  Description of Artificial Sequence metA-rev
cgcgagctcagtactattaatccagcg

SEQ ID NO: 9 - DNA, 37 bp, Artificial Sequence
  Description of Artificial Sequence metJ-fw
gatcgcggccgctgcaacgcggcatcattaaattcga

SEQ ID NO: 10 - DNA, 36 bp, Artificial Sequence
  Description of Artificial Sequence metJ-rev
gatcgcggccgcagtttcaaccagttaatcaactgg